(19) United States
(12) Patent Application Publication
(10) Pub. No.: US 2011/0152630 A1
(43) Pub. Date: Jun. 23, 2011

(54) METHOD FOR RECORDING AND FORWARDING VITAL SIGNS, AS WELL AS DEVICE FOR THIS PURPOSE

(76) Inventors: Hongquan JIANG, Stuttgart (DE); Frank BARTH, Tamm (DE); Christian MAEURER, Aachen (DE)

(21) Appl. No.: 12/959,527

(22) Filed: Dec. 3, 2010

(30) Foreign Application Priority Data
Dec. 4, 2009 (DE) 102009047477.3

(51) Int. Cl. A61B 5/00

(52) U.S. Cl. 600/300

(57) ABSTRACT

In a method for recording and forwarding vital signs of a user, vital signs are measured, possibly linked, and displayed to the user. Once the user has accepted the displayed vital signs or their linkages, the user releases them for transmission by way of an authentification.

### FIELD OF THE INVENTION

The present invention relates to a method for recording and forwarding vital signs of a user.

### BACKGROUND INFORMATION

In telemedical applications, medical data, so-called vital values (vital signs), must be transmitted from a medical measuring device, e.g., a personal scale, sphygmomanometer etc., to a telemedical center. There, the data are processed, evaluated and interpreted. As a rule, the data are initially transmitted from the measuring device to a base station and then forwarded from there via public data and telephone networks such as mobile radio communication, PSTN, IP, to the telemedical center. German Patent No. DE 101 54908 shows such a system.

It is important that the data be clearly assignable to the particular person, that is to say, that the ascertained weight is the weight of the person to be monitored and not that of some other person sharing the home. A clear assignment of the patient is able to be made via an identification of the patient during the measurement.

Most telemedical terminals and/or base stations or telemedical systems do not identify the patient, or they use a disadvantageous method for this purpose. Currently used methods for identifying the patient may be subdivided into two groups: Either a plausibility check is carried out based on the trend of the current measured values, and/or identification is made via biometrical data or codes, such as code cards. One example of such a method is described in U.S. Patent Application No. 2004/0117207.

The first method has the disadvantage that a reliable identification of the patient is not provided. The second methods are frequently complex and time-consuming in their execution, which makes them unsuitable for the clientele in question.

### SUMMARY OF THE INVENTION

With the aid of features according to the present invention, i.e., the vital signs of a user are measured, possibly linked, and displayed to the user, the user releasing them for transmission via an authentification after the user has accepted the displayed vital signs or their linkage, the user has complete control over his or her forwarded data.

In contrast to current methods, where a user first identifies himself prior to the measurement of the vital signs and their processing or linkage, in the method according to the present invention the vital signs to be transmitted or the data derived therefrom are displayed to the user once more in their entirety before the user initiates the release of these data for the transmission of his or her authentification. Nevertheless, a complete decoupling of measurement and the transmission of the data takes place. A measurement may, but need not necessarily, be followed by a data transmission. The user/patient retains full control over his or her measured data.

One shortcoming of current methods is that data are processed immediately following the measurement, although it may be obvious to patients that an error has occurred during the measurement. If the patient becomes aware of an operating error during the measuring operation, or if he does not wish a transmission to take place for other reasons, he is unable to abort the data transmission or the data processing.

In the approach according to the present invention, the transmission of the data turns into a deliberate act on the part of the user/patient. Only through the authentification does the patient agree to the transmission of precisely these data and to their being made available to the system.

Important is that the patient is given a clear and easily readable list of the particular data to be transmitted. Thus, the patient is able to check the plausibility of the data immediately prior to the transmission. The patient transmits the data only after being satisfied with the measuring operation and the measuring result. A further advantage is that it is not the measurement results that are personalized, but that only the transmission is performed in a personalized manner. Until the patient is identified at the device, the data are not patient-specific. The terminal thus always remains a conventional measuring device for which no specific requirements are stipulated. This variant has the advantage that more than one person in the same house or in the same facility is able to use the terminal but that a clear assignment of the measured value and the person is possible nevertheless.

Less desirable is an early linkage of the data and the person. From the moment this linkage is made, the data are linked to the person. At that point, special data privacy rules and requirements must be observed, which increases the demands on the telemedicine system. Ideally, a linkage between patient and data should thus be made only at the end of the data flow path, i.e., in the telemedical center.

As an alternative, it is also possible to realize the entire transmission leading to the telemedical center in a non-personalized manner. For this purpose, it is not the data of the patient that are transmitted but rather simply the identity of the device at which the patient has identified himself. An assignment between authentification unit and patient may then take place with the aid of a table. However, this presupposes that the authentification unit is able to be activated by a particular person only.

In a device according to the present invention for recording and forwarding vital signs of a user, an authentification unit is provided to which the medical measuring devices for measuring the vital signs are able to be connected. It is designed in such a way that the vital signs are transmittable to a medical center only following an authentification by the user, the transmission possibly taking place via an interconnected base station.

The authentification unit advantageously has a display device, which allows the user/patient to make the authentification only after the vital signs have been displayed and possibly linked. This design does not require the measuring devices to have their own display field for the recorded vital signs. Instead, the display of the authentification unit is used as display for all measuring devices.

Furthermore, it is advantageous to buffer-store the vital signs, in particular in a memory of the authentification unit, until all measured values have been recorded and checked by the user, and to initiate the authentification for the transmission only at that stage. This also applies to the linking of the measured values.

It is advantageous to transmit the authentification to a telemedical center together with the vital signs. There, the transmitted vital signs are able to be assigned to a corresponding electronic patient file in a reliable manner.

In one alternative, the device identification of the authentification unit but not the authentification itself is transmitted. The entire transmission leading to the telemedical center is non-personalized in such a case. The assignment between authentification unit and patient then first takes place at the telemedical center, by a chart comparison, for example.

The authentification unit for the authentification is advantageously designed in such a way that it includes at least one sensor for the input of biometrical data, and/or the input of a code or key. This makes the authentification of a patient very simple and reliable and adaptable to the capabilities of the patient with regard to a manual code entry and his or her retentiveness.

### BRIEF DESCRIPTION OF THE DRAWING

FIG. 1 shows a circuit diagram for an authentificated transmission of vital signs, according to the present invention.

### DETAILED DESCRIPTION

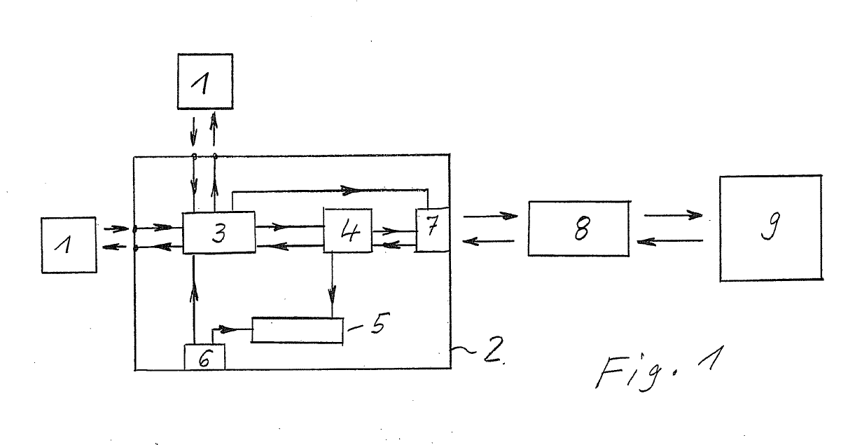

FIG. 1 shows medical measuring devices 1 which are connected to an authentification unit 2. The vital signs recorded by measuring devices 1 are forwarded to authentification unit 2 by a wireless short-range radio transmission or also in wire-bound manner. There, the recorded vital signs are buffer-stored in memory 3, possibly linked in linkage unit 4, and displayed on a display unit 5. The user/patient checks the displayed vital signs. If they seem plausible to the user, then his only option is to authentificate himself at authentification unit 2 and thus agree to the transmission of the data. He may also delete the data or repeat the measuring operation. Following the authentification, the vital signs or their linkages are transmitted to base station 8 with the aid of transmission unit 7.

The performed authentification should be documented there and the authentification information appended to the data or linked to them by an algorithm. This ensures that users, or alternatively authentification unit 2, and the data stay linked at all times. The transmission to the telemedical center then takes place from base station 8. The transmission between the individual units of the system may preferably be verified, e.g., by the exchange of check sums, and take place in a secure manner, such as with the aid of encryption. As one exemplary embodiment, authentification unit 2 may be mounted at eye level of the user/patient above a scale as medical measuring device 1. The vital signs such as weight are displayed on display unit 5 of authentification unit 2 immediately after the weighing operation. The user/patient reviews the value and releases the transmission to base station 8 by finger pressure.

The authentification may be made by checking biometrical data such as a finger print, vessel pattern, voice analysis, detection of movement patterns, recognition of persons based on external optical features. Toward this end, the authentification unit has at least one corresponding sensor 6. Furthermore, the user is also able to identify himself via a code or a key that is carried along. The key may be an electronic key, e.g., an RFID, or a purely mechanical key.

What is claimed is:

1. A method for recording and forwarding vital signs of a user, comprising:
measuring the vital signs;
displaying the vital signs to the user; and
once the user has accepted the displayed vital signs or a linkage of the vital signs, releasing the vital signs by the user for transmission by way of an authentification.

2. The method according to claim 1, further comprising linking the vital signs.

3. The method according to claim 1, further comprising buffer-storing the vital signs until the user releases the vital signs for transmission via an authentification, or until, the user deletes the vital signs.

4. The method according to claim 1, further comprising transmitting the authentification to a telemedical center together with the vital signs.

5. The method according to claim 1, further comprising transmitting a device identification of an authentification unit at which the user has authentificated himself, but not the authentification itself.

6. The method according to claim 1, wherein the authentification takes place by checking biometrical data via a key or a code.

7. A device for recording and forwarding vital signs of a user, comprising:
an authentification unit to which medical measuring devices for measuring vital signs are able to be connected, and from which the vital signs are able to be transmitted to a medical center only following an authentification by the user.

8. The device according to claim 7, wherein the authentification unit communicates with the medical center through a base station.

9. The device according to claim 7, wherein the authentification unit includes a display unit, which allows a user to perform the authentification only following the display of the vital signs and their linkage.

10. The device according to claim 7, wherein the authentification unit includes a memory for determined vital signs, and a linking device for the determined vital signs.

11. The device according to claim 7, wherein a device identification, which is able to be transmitted following the authentification, is stored in the authentification unit.

12. The device according to claim 7, wherein for authentification purposes, the authentification unit includes at least one sensor for an input of at least one of (a) biometrical data, (b) a code and (c) a key.
